(19) United States
(12) Patent Application Publication
(10) Pub. No.: US 2019/0366054 A1
(43) Pub. Date: Dec. 5, 2019

(54) WINGED NEEDLE HUB AND INTERNAL FISTULA NEEDLE SET USING SAME

(71) Applicant: Chih-Hua HUNG, HSINCHU CITY (TW)

(72) Inventor: Chih-Hua HUNG, HSINCHU CITY (TW)

(21) Appl. No.: 16/417,916

(22) Filed: May 21, 2019

(30) Foreign Application Priority Data
Jun. 4, 2018 (TW) 107119193

(51) Int. Cl. A61M 25/06; A61M 39/10; A61M 5/158

(52) U.S. Cl. CPC A61M 25/0637; A61M 25/0618; A61M 25/0097; A61M 5/158; A61M 39/10

(57) ABSTRACT

A winged needle used in an internal fistula need set is made of an elastic material in one piece, having a hub for holding a needle, two wings respectively connected to two opposite sides of the hub and a protective cover connected to the front end of the hub and elastically movable relative to the hub between an open position where the needle is completely exposed for use to inject medicine or to withdraw the blood and a closed position where the needle is shielded by the protective cover to prevent the operator from being accidentally stabbed by the needle during needle collection. Thereby, the winged needle hub of the invention can achieve the advantages of simplified structure, convenient operation and improved safety of the needle.

### BACKGROUND OF THE INVENTION

### 1. Field of the Invention

The present invention relates to fistula needle technology and more particularly, to a winged needle hub and an internal fistula needle set using the winged needle hub.

### 2. Description of the Related Art

The internal fistula needle set is different in structure from the general injection needle and is mainly provided with a wing on the opposite sides of the needle hub connected to the needle. The two wings are used on the one hand to allow the operator to easily hold the injection to complete the injection, and on the other hand to adhere to the skin to stabilize the position of the needle, enabling the medicine to be delivered into the human body, or allowing the blood to be withdrawn from the human body.

In order to ensure that the needle will not be damaged before use and will not stab the operator's finger after use, the structure will be tied with a protective cover for shielding the needle. When an injection is to be performed, the protective cover is opened, allowing the needle to be inserted into the part of the human body to be injected. When the needle is removed from the human body after the injection is done, the protective cover must be capped on again to shield the needle. Various protective cover designs are known. Exemplars are seen in Taiwan Patent Numbers M488318, M494001 and M496494. The protective cover disclosed in the aforementioned patents is not only structurally complicated but must be assembled separately with the winged needle hub, so that the risk of accidental stab wound by the needle may still occur during the assembly process.

### SUMMARY OF THE INVENTION

The present invention has been accomplished under the circumstances in view. It is the main object of the present invention to provide a winged needle hub for internal fistula needle set, which achieves the advantages of simplified structure, convenient operation and improved safety of the needle.

To achieve this and other objects of the present invention, a winged needle hub comprises a hub, two wings and a protective cover. The hub defines a front end and an opposite rear end. The two wings are respectively integrally connected to two opposite sides of the hub. The protective cover comprises a needle groove. Further, the protective cover is connected to the front end of the hub and elastically movable relative to the hub between an open position and a closed position. When the protective cover is in the open position, the central axis of the hub does not pass the needle groove of the protective cover. At this time, the needle that is fixed to the hub is completely exposed for use to inject medicine or to withdraw the blood. When the protective cover is in the closed position, the central axis of the hub does pass the needle groove of the protective cover. At this time, the needle is shielded by the protective cover to prevent the operator from being accidentally stabbed by the needle.

As can be seen from the above, the winged needle hub provided by the present invention is manufactured by an integral molding method. When compared with the discrete design of the conventional techniques, the invention effectively simplifies the structure and reduces the manufacturing cost. When the needle is to be stored, only one finger is needed to complete the operation of the protective cover, thereby greatly reducing the chance of contact between the hand and the needle. Thus, the invention greatly increases the convenience of operation and improves the safety of the needle.

Preferably, one wing comprises a positioning protrusion at a top surface thereof and the other wing comprises a positioning recess at a top surface thereof. When the top surfaces of the two wings are abutted against each other, the positioning protrusion is engaged into the positioning recess to prevent relative movement of the two wings in affecting the stability of the injection process.

Preferably, each wing has an anti-slip pattern at the bottom surface thereof. Thus, the two wings do not slip when they are secured to the skin.

Preferably, the protective cover further comprises an opening and an elastic rib. The elastic rib has one end thereof integrally connected to the peripheral wall of the opening and extends obliquely from the opening toward the needle groove. When the needle is received in the needled groove of the protective cover, the needle is held in position by the elastic rib, preventing accidental separation between the two.

Further, it is another object of the present invention to provide an internal fistula needle set that uses the aforesaid winged needle hub. In addition to the needle and the winged needle hub, the internal fistula needle set further comprises a tubing. The tubing is provided at the rear end of the hub of the winged needle hub to provide the effect of delivering liquid medicine or blood for transferring medicine or blood.

Other advantages and features of the present invention will be fully understood by reference to the following specification in conjunction with the accompanying drawings, in which like reference signs denote like components of structure.

### BRIEF DESCRIPTION OF THE DRAWINGS

FIG. 1 is an oblique top elevational view of a winged needle hub in accordance with the present invention.

FIG. 2 is a bottom view of the winged needle hub in accordance with the present invention.

FIG. 3 is an oblique top elevational view of an internal fistula needle set embodying the present invention, illustrating a protective cover in an open position.

FIG. 4 is similar to FIG. 3, illustrating the top surfaces of the two wings abutted against each other.

FIG. 5 is similar to FIG. 3, illustrating the protective cover in the closed position.

FIG. 6 is a side view of a part of the internal fistula needle set, illustrating the needle positioned in the needle groove and held down by the elastic ribs.

FIG. 7 is a sectional end view of the internal fistula needle set, illustrating the needle positioned in the needle groove and held down by the elastic ribs.

### DETAILED DESCRIPTION OF THE INVENTION

The applicant first describes here, and in the entire specification, including the preferred embodiment and the drawings, the same reference numerals are used to refer to the same or similar elements or structural features thereof.

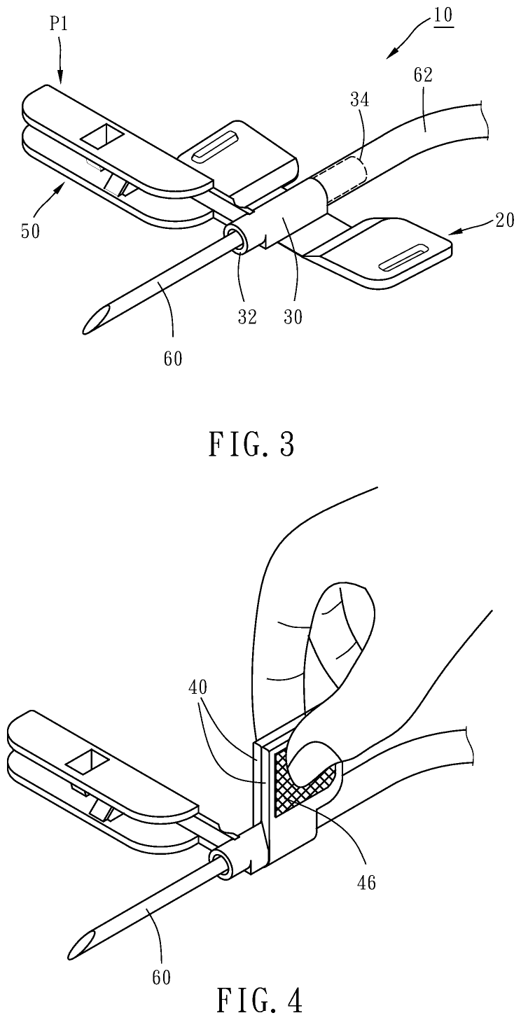

Referring to FIG. 3, an internal fistula needle set 10 in accordance with the present invention comprises a winged needle hub 20, a needle 60 and a tubing 62.

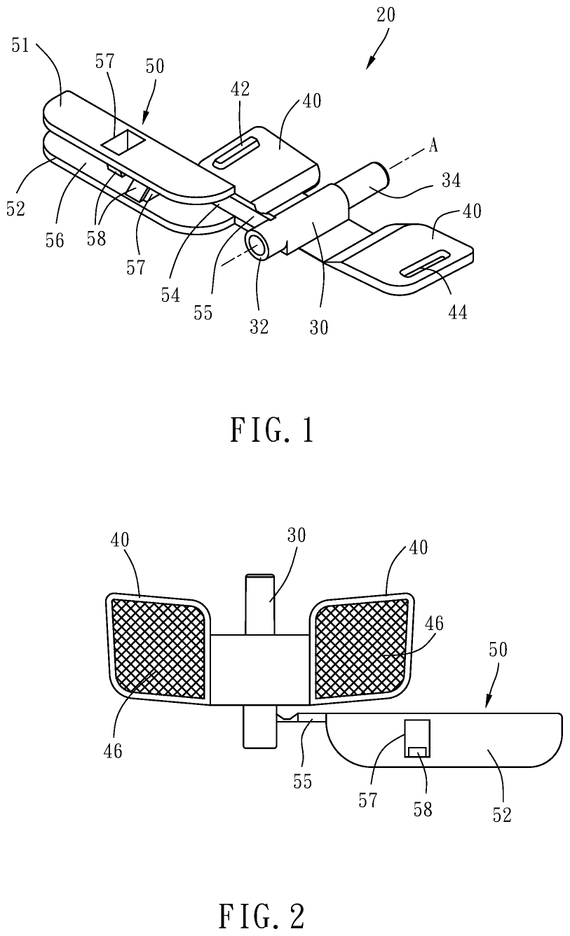

The winged needle hub 20 is made of an elastic material in one piece (plastic injection molding), comprising a hub 30, two wings 40, and a protective cover 50, as shown in FIGS. 1 and 2.

The hub 30 is a hollow tubular member, defining a front end 32 and an opposite rear end 34.

The two wings 40 are respectively connected to opposing left and right sides of the hub 30. One wing 40 has a positioning protrusion 42 located on a top surface thereof. The other wing 40 has a positioning recess 44 located on a top surface thereof. Further, as illustrated in FIG. 2, each wing 40 has an anti-slip pattern 46 on an opposing bottom surface thereof.

The protective cover 50 is connected to a left side of the front end 32 of the hub 30. More specifically, as shown in FIGS. 1, 6 and 7, the protective cover 50 has a top wall 51, a bottom wall 52, an end wall 53, a sidewall 54 and a connection wall 55. The end wall 53 is connected between a front end of the top wall 51 and a front end of the bottom wall 52. The sidewall 54 connects the top wall 51, the bottom wall 52 and a left side of the end wall 53 so as to surround a needle groove 56 which is open to an opposing right side. The connection wall 55 is connected between the front end of the hub 30 and a rear end of the sidewall 54 so that the protective cover 50 is elastically biasable relative to the hub 30. Further, in the present preferred embodiment, as shown in FIGS. 1,2, 6 and 7, the top wall 51 and the bottom wall 52 respectively have an opening 57, and the two openings 57 are staggered one after the other.

The protective cover 50 further has two elastic ribs 58. Each elastic rib 58 has one end thereof connected to the peripheral wall of one respective opening 57 and extends obliquely from the respective opening 57 toward the needle groove 56.

However, it should be added here that the number of openings 57 and elastic ribs 58 can be adjusted according to actual needs, as long as at least one. More specifically, one opening 57 is disposed on one of the top and bottom walls 51,52; one elastic rib 58 has its one end integrally connected to the peripheral wall of the opening 57 and extends obliquely from the opening 57 toward the needle groove 56. This arrangement can also provide a positioning effect.

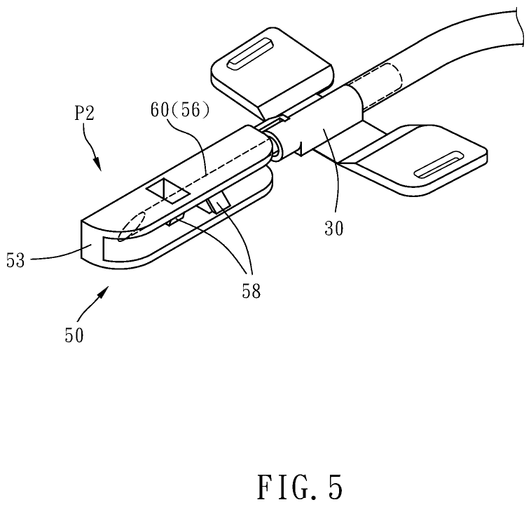

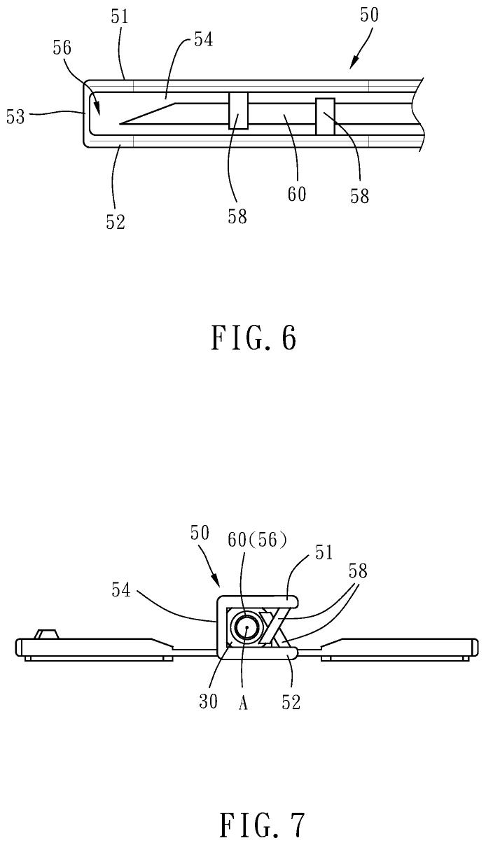

The needle 60 can be fixed to the front end of the hub 30 by means of an integral molding method (such as metal insert molding) or can be fixed to the front end of the hub 30 by an adhesive, which is not limited herein. After the needle 60 is fixed to the hub 30, the needle 60 is located on the central axis A of the hub 30. Thereby, when the protective cover 50 is located at an open position P1 (as shown in FIGS. 1 and 3), the central axis A of the hub 30 does not pass the needle groove 56 of the protective cover 50, so that the needle 60 is completely exposed. When the protective cover 50 is in a closed position P2 (as shown in FIGS. 5 and 7), the central axis A of the hub 30 passes the needle groove 56 of the protective cover 50 so that the needle 60 is located within the needle groove 56 of the protective cover 50, avoiding exposure. Further, when the needle 60 is positioned within the needle groove 56 of the protective cover 50, it is simultaneously positioned by the two elastic ribs 58 as shown in FIGS. 6 and 7, to ensure that the protective cover 50 and the needle 60 are not easily separated.

The tubing 62 sets on the rear end 34 of the hub 30 for the delivery of medicine or blood.

As can be seen from the above, when the injection is to be performed, the protective cover 50 is biased from the closed position P2 as shown in FIG. 5 to the open position P1 as shown in FIG. 3 with the finger, so that the needle 60 is completely exposed. Then, the two wings 40 are pinched by two fingers, as shown in FIG. 4, enabling the top surfaces of the two wings 40 to be abutted against each other. At this time, the positioning protrusion 42 and the positioning recess 44 are engaged together to avoid relative movement of the two wings 40 to affect the stability of the injection process. Then you can insert needle 60 into the part you want to inject. After the insertion of the needle 60 into the part you want to inject, the force applied to the two wings 40 is released, and the two wings 40 are adhered to the skin by using tapes, and the anti-slip patterns 46 of the two wings 40 can be further utilized to stabilize the position of the needle 60. In this way, the medicine can be delivered to the body through the tubing 62, or the blood can be withdrawn from the body of the patient.

After the injection is done, the needle 60 is withdrawn from the body of the patient, and then the protective cover 50 is biased from the open position P1 as shown in FIG. 3 to the close position P2 as shown in FIG. 5 until the needle 60 is located in the needle groove 56 and held in place by the two elastic ribs 58 (as shown in FIGS. 6 and 7).

In summary, the winged needle hub 20 provided by the present invention is manufactured by an integral molding method. When compared with the conventional techniques, the invention effectively simplifies the structure and reduces the manufacturing cost. When the needle 60 is to be stored, only one finger is needed to complete the operation of the protective cover 50, thereby greatly reducing the chance of contact between the hand and the needle 60. Thus, the invention greatly increases the convenience of operation and improves the safety of the needle.

What is claimed is:

1. A winged needle hub for internal fistula needle set, comprising:
a hub defining a front end and an opposite rear end;
two wings respectively integrally connected to two opposite sides of said hub; and
a protective cover comprising a needle groove, said protective cover being integrally connected to said front end of said hub and elastically movable relative to said hub between an open position where said needle groove of said protective cover is disposed beyond a central axis of said hub and a closed position where said central axis of said hub passes said needle groove of said protective cover.

2. The winged needle hub as claimed in claim 1, wherein said hub, said two wings and said protective cover are integrally made of an elastic material in one piece.

3. The winged needle hub as claimed in claim 1, wherein one said wing comprises a positioning protrusion at a top surface thereof and the other said wing comprises a positioning recess at a top surface thereof, said positioning protrusion being engaged into said positioning recess when the top surfaces of said two wings are abutted against each other.

4. The winged needle hub as claimed in claim 1, wherein each said wing comprises an anti-slip pattern on an opposing bottom surface thereof.

5. The winged needle hub as claimed in claim 1, wherein said protective cover comprises a top wall, a bottom wall, an end wall, a sidewall and a connection wall, said end wall being integrally connected between a front end of said top wall and a front end of said bottom wall, said sidewall connecting said top wall, said bottom wall and one side of said end wall to surround a needle groove, said connection wall being integrally connected between the front end of said hub and a rear end of said sidewall.

6. The winged needle hub as claimed in claim 5, wherein said protective cover further comprises an opening and an elastic rib, said opening being located one of the said top wall and said bottom wall of said protective cover, said elastic rib having one end thereof integrally connected to the peripheral wall of said opening and extending obliquely from said opening toward said needle groove.

7. The winged needle hub as claimed in claim 5, wherein said protective cover further comprises two openings and two elastic ribs, said two openings being respectively located on said top wall and said bottom wall and staggered one after the other, each said elastic rib having one end thereof integrally connected to the peripheral wall of one respective said opening and extending obliquely from the respective said opening, said two elastic ribs being extended in reversed directions.

8. An internal fistula needle set, comprising:
a winged needle hub as claimed in claim 1;
a needle set on said front end of said hub of said winged needle hub and located on the said central axis of said hub of said winged needle hub, said needle being disposed outside said needle groove of said protective cover when said protective cover of said winged needle hub is in said open position, said needle is disposed in said needle groove of said protective cover when said protective cover of said winged needle hub is in said closed position; and
a tubing fastened to the said rear end of said needle hub.

9. The internal fistula needle set as claimed in claim 8, wherein said winged needle hub is integrally made of an elastic material in one piece.

10. The internal fistula needle set as claimed in claim 8, wherein one said wing of said winged needle hub comprises a positioning protrusion at a top surface thereof and the other said wing of said winged needle hub comprises a positioning recess at a top surface thereof, said positioning protrusion being engaged into said positioning recess when the top surfaces of said two wings are abutted against each other.

11. The internal fistula needle set as claimed in claim 8, wherein each said wing of said winged needle hub comprises an anti-slip pattern on an opposing bottom surface thereof.

12. The internal fistula needle set as claimed in claim 8, wherein said protective cover of said winged needle hub comprises a top wall, a bottom wall, an end wall, a sidewall and a connection wall, said end wall being integrally connected between a front end of said top wall and a front end of said bottom wall, said sidewall connecting said top wall, said bottom wall and one side of said end wall to surround a needle groove, said connection wall being integrally connected between the front end of said hub and a rear end of said sidewall.

13. The internal fistula needle set as claimed in claim 12, wherein said protective cover of said winged needle hub further comprises an opening and an elastic rib, said opening being located one of the said top wall and said bottom wall of said protective cover, said elastic rib having one end thereof integrally connected to the peripheral wall of said opening and extending obliquely from said opening toward said needle groove; said needle is positioned in said needle groove of said protective cover and held in position by said elastic rib when said protective cover is in said closed position.

14. The internal fistula needle set as claimed in claim 12, wherein said protective cover of said winged needle hub further comprises two openings and two elastic ribs, said two openings being respectively located on said top wall and said bottom wall and staggered one after the other, each said elastic rib having one end thereof integrally connected to the peripheral wall of one respective said opening and extending obliquely from the respective said opening, said two elastic ribs being extended in reversed directions; said needle is positioned in said needle groove of said protective cover and held in position by said elastic ribs when said protective cover is in said closed position.

15. The internal fistula needle set as claimed in claim 8, wherein said needle is integrally connected to said front end of said hub.

16. The internal fistula needle set as claimed in claim 8, wherein said needle is affixed to said front end of said hub using an adhesive.
